(19) United States
(12) Patent Application Publication
(10) Pub. No.: US 2015/0190332 A1
(43) Pub. Date: Jul. 9, 2015

(54) MULTI-LAYER COSMETIC FILMS

(71) Applicant: Avon Products, Inc., Suffern, NY (US)

(72) Inventors: Ashley L. Howell, Oakland, NJ (US); Michael J. Fair, Ridgewood, NJ (US); John R. Glynn, JR., Ridgewood, NJ (US); Amanda R. Stagg, Morris Plains, NJ (US)

(21) Appl. No.: 14/408,985

(22) PCT Filed: Mar. 5, 2014
(86) PCT No.: PCT/US14/20528

§ 371 Date: Dec. 18, 2014

Related U.S. Application Data
(60) Provisional application No. 61/792,628, filed on Mar. 15, 2013.

(51) Int. Cl. A61K 8/81; A61Q 1/02; A61K 8/29

(52) U.S. Cl. CPC A61K 8/8152; A61K 8/29; A61Q 1/02; A61K 2800/262

(57) ABSTRACT

Cosmetic compositions are provided, which are capable of forming an immiscible multi-layered composition. Cosmetic compositions according to the invention typically include a first composition comprised of a pH dependent film-forming polymer with a target pH and wherein the pH of the first composition is greater than the target pH, and a second composition having a pH less than or equal to the target pH. The immiscible multi-layered composition enable various optical effects that provide enhance coverage, color trueness and enhanced naturalness.

This application claims priority benefit to U.S. Provisional Patent Application Ser. No. 61/792,628, filed on Mar. 15, 2013, the entirety of which is herein incorporated by reference for all purposes.

### FIELD OF THE INVENTION

The present application generally relates to cosmetic compositions that provide increased optical benefits and unique colors and textures. More particularly, the application relates to an immiscible multi-layered cosmetic composition. The composition has a base or primer layer having a pH-dependent polymer and a pH greater than the target pH of the pH-dependent polymer, the pH at which the pH dependent polymer becomes soluble. A top layer, having a pH less than the target pH of the pH-dependent polymer, is applied over the base or primer coat and is immiscible therewith. Unique or enhanced optical effects may be obtained through the inclusion of particles and or pigments within the base and/or top layers.

### BACKGROUND OF THE INVENTION

There is an increasing demand in the cosmetics industry to develop products that may be applied topically to improve the appearance of skin while maintaining the skin's natural appearance. Ideally, a biological surface, such as skin, would have a translucent appearance with uniform coloring, and a smooth and even surface with no apparent imperfections.

Few consumers naturally possess skin with this ideal appearance. There is great interest in covering or mitigating dermatological blemishes, imperfections and signs of chronological, hormonal, or environmental aging such as fine lines, wrinkles, drying, acne, scars, and sagging skin. Therefore, there is a need for cosmetics that assist in creating a flawless, natural look.

Currently there is a technology gap in topical skin and color cosmetic products. There exists a need for a composition which will efficiently blur fine lines and wrinkles, as well as provide the required hiding to cover discolorations, age spots, and blemishes to match skin tone with the natural look of young skin. Simultaneous blurring and coverage is difficult to achieve because cosmetic ingredients which provide the desired color and coverage, are generally opaque and tend to obscure the vibrant and natural translucency of the skin. Pigments or particles with less opacity are available, but they may not provide enough coverage to mask or camouflage the flaws in or on the biological surface, including skin. Traditionally, cosmetic primers and concealers have been used to conceal the above-noted imperfections. However, the primers and concealers tend to blend with whatever make-up is used as a topcoat. This decreases the efficacy of the primer/concealer and defeats the purpose of application to achieve higher coverage, lastingness, etc. Further, two layered cosmetic compositions having a liquid base layer and a powder top layer have been used to provide a natural look but the top coat of the powder often provides an undesirable powdery appearance.

There is a need for a system of cosmetic compositions and method of use thereof to improve the aesthetic appearance of skin. Therefore, safe and effective systems of compositions that provide a translucent appearance with uniform coloring, and smooth and even biological surfaces with no apparent imperfections would be advantageous for the formulation of treatments and products for biological surfaces, including the skin.

U.S. Patent Publication No. 2009/0148393 to Maitra et al. discloses a multi-step cosmetic composition having a pigmented base coat and a diffused topcoat.

U.S. Patent Publication No. 2010/0266517 to Dingley et al. discloses a two-coat cosmetic composition that uses a hydrophilic base coat and a top coat to form a hydrophobic cosmetic film on a biological surface. The incompatibility of the hydrophilic base coat and the top coat attribute to the water-resistant, oil-resistant, migration-resistant, smooth and durable nature of the formed film.

U.S. Patent Publication No. 2005/0129649 to Kurosawa et al. discloses the use of a liquid foundation of silicone and silicone-based polysaccharides and a finishing powder with diffuse reflection powder to provide a natural appearance.

Despite advances in film-forming methods and compositions, there still remains a need for cosmetic compositions that consistently provide a natural and aesthetically appealing or flawless appearance.

### SUMMARY OF THE INVENTION

It has surprisingly been found that certain cosmetic compositions incorporating a first layer having a pH-dependent film-forming polymers having a target pH and a second layer having a pH less than or equal to the target pH are able to form immiscible multilayered films that enable enhanced optical effects.

In a method of the current invention, a first cosmetic composition and a second cosmetic composition are applied to a human integument to obtain a benefit. The first cosmetic composition has a pH dependent film forming polymer having a target pH, a cosmetically or pharmaceutically acceptable vehicle, and a pH greater than the target pH. The second cosmetic composition has a cosmetically or pharmaceutically acceptable vehicle and a pH less than or equal to the target pH. According to the method, a basecoat of the first cosmetic composition to the human integument so as to form an immiscible base layer, and then a topcoat of the second cosmetic composition is applied to the basecoat. The first cosmetic and the second cosmetic are applied in amounts sufficient to achieve the desired enhancement of the human integument.

In a further embodiment of the above-noted method, the method provides the benefit of enhanced coverage. The first cosmetic composition further includes at least one particle or powder and the first cosmetic composition and the second cosmetic composition are applied in amounts to achieve enhanced coverage of at least 50% (in one embodiment, at least 52%; in another embodiment at least 54%; in another embodiment, at least 56%; in another embodiment, at least 58%) and diffuse transmittance of no less than 20% (in one embodiment, no less than 30%; in another embodiment, no less than 40%; in another embodiment, no less than 42%; in another embodiment, no less than 44%; in another embodiment, no less than 46%; in another embodiment, no less than 48%). In a further embodiment of this particular method, the at least one particle or pigment may be present in about 1-20% of the first composition. Additionally, at least one particle or pigment may be titanium dioxide.

In yet another embodiment, the method enhances the color trueness of a pigment applied to the integument. The first cosmetic composition further includes at least one particle, powder or pigment having a refractive index greater than or equal to about 1.40, and the second cosmetic composition also includes at least one pigment. The first and second cosmetic compositions are applied in amounts sufficient to improve the color trueness of the pigment applied to the human integument. In certain embodiments of this method, the particle, powder, or pigment having a refractive index greater than or equal to about 1.40 may be present in the first composition in about 1-15%.

In still another embodiment of the above-noted method, the first cosmetic composition also includes at least one soft focus particle or powder, and the second cosmetic includes at least one transparent pigment. Both the first and second cosmetic compositions are applied in amounts sufficient to improve the naturalness, provide diffuse transmittance of at least 40%, of the human integument. In a further embodiment, the transparent pigment may be a transparent yellow oxide or transparent red oxide or transparent brown oxide or transparent black oxide, and in a further embodiment the transparent pigment may be an acicular titanium dioxide.

In yet a further embodiment of the method, the first cosmetic composition includes at least one active, and the first and second cosmetic compositions are applied in an amount sufficient to provide a benefit to the human integument. The active may be a retinoid in a further embodiment of this invention.

In a further embodiment of the above-noted methods, the human integument may be skin.

The pH-dependent film-forming polymer of the above-noted methods may be a polymer of poly(methacrylic acid-co-methyl methacrylate) with a ratio of methacrylic acid to methyl methacrylate of about 1:1 to about 1:2, an acid value of from about 150 to about 350 mg KOH/g, and a weight average molar mass between about 100,000 and about 150,000 g/mol in certain embodiments. In a further embodiment, the ratio of methacrylic acid to methyl methacrylate may be about 1:1 for this polymer. The ratio of methacrylic acid to methyl methacrylate is about 1:2 in yet another embodiment. The acid value of the polymer may be about 150 to about 200 mg KOH/g in a further embodiment, and from about 300 to about 350 mg KOH/g in yet another embodiment. In one embodiment, the pH dependent polymer of the methods noted above has a target pH of greater than about 6, and a target pH of greater than about 7 in a further embodiment.

In a further embodiment of the above-noted methods the pH-dependent film forming polymer is a polymer of poly(methacrylic acid-co-ethyl acrylate) with a ratio of methacrylic acid to ethyl acrylate of about 1:1, an acid value of from about 300 to about 350 mg KOH/g, and a weight average molar mass between about 200,000 and about 350,000 g/mol. In one embodiment, this pH dependent film-forming polymer has a target pH of greater than about 5.5.

In yet another embodiment of the methods, the pH-dependent film-forming polymer may be present in an amount of about 0.1 wt % to about 15% of the total weight of the composition of the first composition, in an amount of about 0.2 wt % to about 8.0% of the total weight of the first composition, and in an amount of about 0.5 wt % to about 5.0% of the total weight of the first composition in further embodiments.

In certain embodiments of the above noted methods, the pH-dependent film forming polymer may not be in the form of a microcapsule.

A further embodiment of the current invention relates to a layered arrangement of cosmetic compositions. The arrangement includes a human integument, a first composition, and a second composition. The first composition is formed as a basecoat contacting the substrate and the second composition is formed as a topcoat contacting the first composition. The first composition may be an immiscible layer with a pH dependent film forming polymer having a target pH, and at least one particle or powder. The second composition has at least one transparent pigment and a pH less than equal to the target pH. The layered arrangement provides a diffuse transmittance of greater than 40%. In further embodiments, the diffuse transmittance is greater than 50%. In yet a further embodiment the transparent pigments of the second cosmetic composition may include a transparent yellow oxide, transparent red oxide, transparent brown oxide, or titanium dioxide.

Another arrangement comprising a human integument, a first composition, and a second composition is a further embodiment of the current invention. The first composition is formed as a basecoat contacting the substrate and the second composition is formed as a topcoat contacting the first composition. The first composition includes an immiscible layer containing a pH dependent film forming polymer having a target pH and at least one particle or powder. The second composition has a pH less than equal to the target pH. The layered arrangement provides coverage of at least about 50% and diffuse transmittance of no less than 40%.

Another embodiment of the current invention, is directed to a kit including a first cosmetic composition comprised of a cosmetically or pharmaceutically acceptable vehicle, a pH-dependent film-forming polymer with a target pH, at least one particle, powder or pigment having a refractive index greater than or equal to about 1.40, and a pH greater than the target pH, and a second cosmetic composition that includes a cosmetically or pharmaceutically acceptable vehicle, at least one pigment, and has a pH less than or equal to the target pH. Further kits, including the first cosmetic compositions and second cosmetic compositions of the above-noted methods may also be included as embodiments herein.

These and other aspects of the invention will be better understood by reading the following detailed description and appended claims.

### BRIEF DESCRIPTION OF THE DRAWINGS

FIGS. 1A and 1B show images obtained from a fluorescence microscope (10× magnification, FITC filter) of VitroSkin coated with a control concealer (no Eudragit) and a foundation, FIG. 1A, and a concealer containing Eudragit (4% in water phase) and foundation, FIG. 1B. These figures illustrate the immiscibility of the Eudragit concealer and foundation in comparison to the control concealer and foundation. In particular, FIG. 1B shows a clear distinction between the Eudragit and non-Eudragit layers, a level of distinction that is lacking in the FIG. 1A depiction of two non-Eudragit layers.

FIG. 2 is a bar graph illustrating the comparative coverage provided by (1) foundation alone, (2) foundation with a layer of control concealer underneath, and (3) foundation with a layer of Eudragit concealer underneath. The graph clearly illustrates superior coverage is provided by the multi-layer cosmetic of the Eudragit concealer and foundation.

FIGS. 3A and 3B are bar charts comparing the L*, a*, and b* values for a single colored cosmetic (lip color) across three Leneta cards representing three different skin types as a measure for color trueness. FIG. 3A illustrates the lip color readings in the absence of a primer layer, and FIG. 3B illustrates the lip color readings with an immiscible primer layer. FIG. 3B shows consistency of color across the three skin types.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Further, the figures are not necessarily to scale, and some features may be exaggerated to show details of one embodiment's components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

The immiscible multi-layered cosmetic films of the current invention provide novel optical effects, such as increased coverage, soft focus/naturalness, and color travel. The base immiscible layer can be applied as a primer or concealer in which subsequent layers of makeup and/or skin care products can be applied on top without disturbing the first layer. This allows for modification of light travel, light transmittance and reflectance, to improve the appearance/overall look of underlying skin.

The immiscible multilayered cosmetic composition of the current invention permits the base layer, initial coat closest to the skin to remain distinct from top layer, layer applied directly or indirectly over the base layer. The distinct layers improve the optical benefits of the base and allow for a more natural coverage with foundation or concealer applied thereon. The multi-layered effect of base and foundation could provide improved naturalness with coverage, allowing for blurring of fine lines and wrinkles, while also covering imperfections. A primer or initial layer containing soft focus materials of varying shapes and sizes may provide the desired blurring effect, and the second layer of foundation or concealer containing pigments may provide the desired coverage of imperfections.

Additionally, the performance of makeup or other cosmetics applied on top of an immiscible base layer can be enhanced. The immiscible base layer can enhance the coverage and color trueness (e.g., coverage). Non-blendable basecoats can also be utilized for color trueness (i.e., to provide coverage of the skin and/or lip tone which can interfere with the thin-film appearance of a color cosmetic on skin/lips vs. its appearance in bulk).

The cosmetic compositions may be in the form of suspensions; serums; lotions; aqueous, dilute alcoholic gels; dispersions; emulsions (e.g., oil-in-water emulsions, water-in-oil emulsions, water-in-silicone emulsions, silicone-in-water emulsions); liquids, and the like. As used herein, the term liquid is intended to include very thin to very viscous materials including non-Newtonian liquids having high initial viscosities (e.g., up to about 2,000,000 cps at 25° C.), as well as gels and other materials capable of dispensation from a container onto a human integument. Preferred cosmetics include, without limitation, mascara, foundation, concealer, face primer, sunscreen, press powder, eye shadow, aqueous lip products (e.g., lipstick and lip-gloss), skin cream, skin gel, and the like.

All terms used herein are intended to have their ordinary meaning in the art unless otherwise provided. All concentrations are in terms of percentage by weight of the specified component relative to the entire weight of the cosmetic composition. Unless otherwise defined, the phrase “substantially free” refers to an amount of a component that is sufficiently low such that the component contributes no significant properties to the bulk and, in any event, will be less than 0.5% by weight and preferably less than 0.1% by weight.

As used herein, the term “consisting essentially of” is intended to limit the invention to the specified materials or steps and those materials or steps that do not materially affect the basic and novel characteristics of the claimed invention, as understood from a reading of this specification. All percentages are by weight based on the total weight of the composition, unless otherwise indicated.

By “cosmetically acceptable” it is meant that a particular component is generally regarded as safe and nontoxic at the levels employed.

### Immiscible Multi-Layer Cosmetic Composition

The immiscible multilayer cosmetic compositions of the current invention are comprised of (1) a base or primer layer incorporating a pH-dependent, film-forming polymer having a pH greater than the target pH of the pH-dependent, film-forming polymer; and (2) a top coat layer having a pH lower than the target pH of the pH-dependent, film-forming polymer incorporated into the base layer.

### A. Base or Primer Coat.

The base or primer coat of the current immiscible multi-layered cosmetic incorporates a pH-dependent film forming polymer. Generally, these polymers comprise anionic copolymers with monomers comprised of acrylic acid/acrylic acid derivatives. In particular, the copolymers may consist of methacrylic acid and methyl methacrylate or ethyl acrylate copolymers in a random, block, or alternating configuration. These polymers have a trigger pH, a pH at which the polymers shift from being insoluble to soluble. These polymers are insoluble in water at a pH below their trigger pH due to non-ionized (i.e., protonated) carboxylic acid groups. However, as the pH of a solution is increased to the trigger pH, the polymers experience ionization of the carboxylic acid group, resulting in increased aqueous solubility and consequent dissolution of the polymer.

In certain embodiments of the invention, the pH-dependent film-forming polymer typically has a molar ratio of methacrylic acid to ethyl acrylate of about 1:1, an acid value of from about 150 to about 350 mg KOH/g, and a weight average molar mass between about 200,000 and about 350,000 g/mol. Typically the pH-dependent film forming polymer may have the following structure

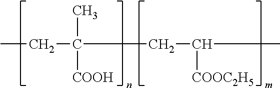

wherein m and n are integers so that the weight average molar mass is between about 200,000 and about 350,000 g/mol and so that the molar ratio of methacrylic acid to ethyl acrylate is about 1:1. Examples of these polymers are available as EUDRAGIT® L 100-55 from Evonik Degussa-Huls Corporation.

In further embodiments of the invention, the pH-dependent film-forming polymer typically has a molar ratio of methacrylic acid to methyl methacrylate of about 1:1 to about 1:2, an acid value of from about 150 to about 350 mg KOH/g, and a weight average molar mass between about 100,000 and about 150,000 g/mol. These pH-dependent film forming polymers may have the following structure

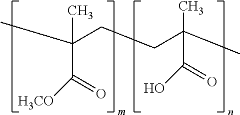

wherein m and n are integers so that the weight average molar mass is between about 100,000 and about 150,000 g/mol and so that the molar ratio of methacrylic acid to methyl methacrylate is about 1:1 to about 1:2. Examples of these polymers are available as EUDRAGIT® S 100 and EUDRAGIT® L 100 from Evonik Degussa-Huls Corporation.

Exemplary pH-dependent polymers according to the invention will comprise methacrylic acid and methyl methacrylate or ethyl acrylate copolymers in a molar ratio from about 1:1 to about 1:2 (e.g., about 1:1, and about 1:2). The polymers typically comprise an acid value of about 150 to about 350 mg KOH/g, including exemplary ranges such as, but not limited to, about 150 to about 200 mg KOH/g and about 300 KOH/g to about 350 KOH/g. Acid values of about 190 mg KOH/g and about 315 mg KOH/g are particularly preferred. Moreover, in certain embodiments, the methacrylic co-methyl methacrylate pH-dependent polymers will typically have an average molar mass from about 100,000 to about 150,000 g/mol, and most typically about 125,000 g/mol. In other embodiments, the methacrylic co-ethyl acrylate pH-dependent polymers will typically have an average molar mass from about 200,000 to about 350,000 g/mol, and most typically about 250,000 g/mol.

The preferred pH-dependent film-forming polymers are sold under the names EUDRAGIT® S 100, EUDRAGIT® L 100, and EUDRAGIT® L100-55 by Evonik Degussa-Huls Corporation. Films or coatings of such polymers are insoluble at pH values less than about 7 (EUDRAGIT® S 100), less than about 6 (EUDRAGIT® L 100), and less than about 5.5 (EUDRAGIT® L 100-55). Accordingly, these polymers may be included in cosmetic compositions either alone or in combination, such that the pH-dependent solubility of a film produced thereby may be selected to be from about 5.5 to about 7, including but not limited to about 5.5, 5.6, 5.7, 5.8, 5.9, 6.0, 6.1, 6.2, 6.3, 6.4, 6.5, 6.6, 6.7, 6.8, 6.9 and 7.0. Such selection will typically be based on the pH of the desired top coat layer to be used in the immiscible multi-layered composition of the current invention. Thus, if the cosmetic intended as a top layer has a pH of about 6, the pH-dependent film-forming polymer should have a trigger pH that is greater than or equal to 6. However, it may be broadly stated that the pH-dependent, film-forming polymers will be selected such that the resulting film will remain insoluble upon application of the top coat. Accordingly, the film-forming polymers may be chosen such that the cosmetic composition is soluble at a pH greater than about 5.0, greater than about 5.5, greater than about 6.0, greater than about 6.5, greater than about 7.0, or greater than about 7.5, depending on the intended use. In certain, embodiments the pH dependent film-forming polymer shall have a target pH of 7.0.

The amount of pH-dependent polymer(s) present in the base coat will typically range from about 0.1 to about 15% by weight of the composition, but may be higher or lower depending on the desired properties. Typically, the base coat compositions will comprise the pH-dependent polymers in an amount of from about 0.1% to about 15% by weight, more typically from about 0.2 to about 8% by weight, and most typically from about 0.5 to about 5% by weight.

The base coat composition itself will have a pH greater than the incorporated pH-dependent film-forming polymer. This permits the base coat composition to be administered to the skin. When applied to the skin, the acidic nature of the skin (pH of about 4.7, Natural skin surface pH is on average below 5, which is beneficial for its resident flora, Int J Cosmet Sci. 2006 October; 28(5):359-70.) adjusts the pH of the base coat composition below the trigger pH of the pH-dependent film-forming polymer, resulting in the formation of an insoluble film. Thus, if the pH-dependent film-forming polymer has a target pH of 5.5 the base coat composition will have a pH greater than 5.5; if the polymer has a target pH of 6.0 the base coat composition will have a pH greater than 6; and so on.

It has been found that cosmetic compositions comprising such pH-dependent polymers are both easy to apply to and remove from the skin, yet remain in contact with the skin for long periods of time, i.e. do not rub-off. See U.S. patent application Ser. No. 13/325,670, filed Dec. 14, 2011 entitled “Long-Lasting Easy Wash-off Cosmetic Compositions. Cosmetic compositions comprising pH-dependent polymers may remain on the skin for greater than about 4, about 6, about 12, about 24, about 48, or even greater than about 72 hours.

The pH film-forming polymer is not in the form of an encapsulant, and is particularly not utilized such as those described in U.S. Pat. Nos. 7,053,034, 4,976,961, 5,993,831 and U.S. Patent App. Pub. No. 2006/0127427. As used herein, the term “microcapsule” refers to a structure having a polymeric membrane (i.e., shell) surrounding a core material (e.g., an active ingredient). The term “microcapsule” is intended to be generic, and is not limited to a particular size (i.e., nano, micro, etc.).

### B. Top/Secondary Layer

The top/secondary layer of the current immiscible multi-layer cosmetic composition may be comprised of a cosmetic composition having a pH equal to or less than the trigger pH of the pH-dependent film-forming polymer of the current invention. For example, if the pH dependent film-forming polymer of the base layer has a target pH of 7, then the top/secondary layer shall have a pH less than or equal to 7.

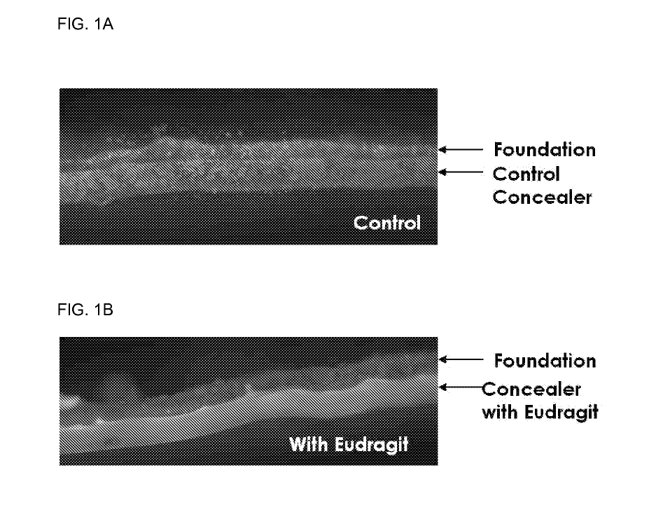

As noted above, the two layers, the base layer and top layer, should be immiscible and this can be confirmed using the fluorescein protocol detailed below in Example 1. FIG. 1B illustrates that when the pH of the top/second layer is maintained below the target pH of the pH dependent film-forming polymer the layers remain distinct and do not mix, i.e. are immiscible. It is through this separation of the base and top layers of the cosmetic composition that the enhanced and novel optical aspects of this invention are possible.

The compositions, base layer and top layer, can include a cosmetically acceptable vehicle. Such vehicles may take the form of any known in the art suitable for application to skin and may include water (e.g., deionized water); vegetable oils; mineral oils; esters such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone crosspolymer, polysiloxanes and their derivatives, such as organomodified derivatives; hydrocarbon oils such as mineral oil, petrolatum, isoeicosane and polyisobutene; polyols such as propylene glycol, glycerin, butylene glycol, pentylene glycol and hexylene glycol; waxes such as beeswax, botanical waxes, carnauba, ozokerite; hydrocarbon waxes such as polyethylene; or any combinations or mixtures of the foregoing.

The vehicle may comprise an aqueous phase, an oil phase, an alcohol, a silicone phase or mixtures thereof. The cosmetically acceptable vehicle may also comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. The emulsion may include an emulsifier, such as a nonionic, anionic or amphoteric surfactant.

The aqueous phase of the emulsion in one embodiment has one or more organic compounds, including emollients; humectants (such as butylene glycol, propylene glycol, Methyl gluceth-20, and glycerin); other water-dispersible or water-soluble components including thickeners such as SepiMAX™ ZEN (Seppic, Puteaux, France), ARISTOFLEX® AVC (Clariant Corporation, Charlotte, N.C.), veegum or hydroxyalkyl cellulose; high MW polyacrylic acid, i.e. CARBOPOL 934; and mixtures thereof. The emulsion may have one or more emulsifiers capable of emulsifying the various components present in the composition.

The compounds suitable for use in the oil phase include without limitation, vegetable oils; esters such as octyl palmitate, isopropyl myristate and isopropyl palmitate; ethers such as dicapryl ether; fatty alcohols such as cetyl alcohol, stearyl alcohol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; natural or synthetic waxes; and the like. Suitable hydrophobic hydrocarbon oils may be saturated or unsaturated, have an aliphatic character and be straight or branched chained or contain alicyclic or aromatic rings. The oil-containing phase may be composed of a singular oil or mixtures of different oils.

Hydrocarbon oils including those having 6-20 carbon atoms may be utilized, and in one embodiment they may have 10-16 carbon atoms. Representative hydrocarbons include decane, dodecane, tetradecane, tridecane, and C₈₋₂₀ isoparaffins. Paraffinic hydrocarbons are available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the tradename Permethyl 99 ATM are also contemplated to be suitable. Various commercially available C₁₆ isoparaffins, such as isohexadecane (having the tradename Permethyl®) are also suitable. Examples of volatile hydrocarbons include polydecanes such as isododecane and isodecane, including for example, Permethyl-99A (Presperse Inc.) and the C₇-C₈ through C₁₂-C₁₅ isoparaffins such as the Isopar Series available from Exxon Chemicals. A representative hydrocarbon solvent is isododecane.

The oil phase may comprise one or more waxes, including for example, rice bran wax, carnauba wax, ouricurry wax, candelilla wax, montan waxes, sugar cane waxes, ozokerite, polyethylene waxes, Fischer-Tropsch waxes, beeswax, microcrystalline wax, silicone waxes, fluorinated waxes, and any combination thereof.

Non-limiting emulsifiers include emulsifying waxes, emulsifying polyhydric alcohols, polyether polyols, polyethers, mono- or di-ester of polyols, ethylene glycol mono-stearates, glycerin mono-stearates, glycerin di-stearates, silicone-containing emulsifiers, soya sterols, fatty alcohols such as cetyl alcohol, acrylates, fatty acids such as stearic acid, fatty acid salts, and mixtures thereof. Emulsifiers may include soya sterol, cetyl alcohol, stearic acid, emulsifying wax, acrylates, silicone containing emulsifiers and mixtures thereof. Other specific emulsifiers that can be used in the composition of the present invention include, but are not limited to, one or more of the following: C₁₀₋₃₀ alkyl acrylate crosspolymer; Dimethicone PEG-7 isostearate, acrylamide copolymer; mineral oil; sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the INCI Ingredient Dictionary and Handbook 11^{th} Edition 2006, the disclosure of which is hereby incorporated by reference in its entirety.

These emulsifiers typically will be present in the composition in an amount from about 0.001% to about 10% by weight, in particular in an amount from about 0.01% to about 5% by weight, and in one embodiment, from about 0.1% to about 3% by weight.

The oil phase may comprise one or more volatile and/or non-volatile silicone oils. Volatile silicones include cyclic and linear volatile dimethylsiloxane silicones. In one embodiment, the volatile silicones may include cyclodimethicones, including tetramer (D₄), pentamer (D₅), and hexamer (D₆) cyclomethicones, or mixtures thereof. Particular mention may be made of the volatile cyclomethicone-hexamethyl cyclotrisiloxane, octamethyl-cyclotetrasiloxane, and decamethyl-cyclopentasiloxane. Suitable dimethicones are available from Dow Corning under the name Dow Corning 200® Fluid and have viscosities ranging from 0.65 to 600,000 centistokes or higher. Suitable non-polar, volatile liquid silicone oils are disclosed in U.S. Pat. No. 4,781,917. Additional volatile silicones materials are described in Todd et al., “Volatile Silicone Fluids for Cosmetics”, Cosmetics and Toiletries, 91:27-32 (1976). Linear volatile silicones generally have a viscosity of less than about 5 centistokes at 25° C., whereas the cyclic silicones have viscosities of less than about 10 centistokes at 25° C. Examples of volatile silicones of varying viscosities include Dow Corning 200, Dow Corning 244, Dow Corning 245, Dow Corning 344, and Dow Corning 345, (Dow Corning Corp.); SF-1204 and SF-1202 Silicone Fluids (G.E. Silicones), GE 7207 and 7158 (General Electric Co.); and SWS-03314 (SWS Silicones Corp.). Linear, volatile silicones include low molecular weight polydimethylsiloxane compounds such as hexamethyldisiloxane, octamethyltrisiloxane, decamethyltetrasiloxane, and dodecamethylpentasiloxane, to name a few.

Non-volatile silicone oils will typically comprise polyalkylsiloxanes, polyarylsiloxanes, polyalkylarylsiloxanes, or mixtures thereof. Polydimethylsiloxanes are non-volatile silicone oils. The non-volatile silicone oils will typically have a viscosity from about 10 to about 60,000 centistokes at 25° C., in one embodiment between about 10 and about 10,000 centistokes, and in one embodiment still between about 10 and about 500 centistokes; and a boiling point greater than 250° C. at atmospheric pressure. Non limiting examples include dimethyl polysiloxane (dimethicone), phenyl trimethicone, and diphenyldimethicone. The volatile and non-volatile silicone oils may optionally be substituted with various functional groups such as alkyl, aryl, amine groups, vinyl, hydroxyl, haloalkyl groups, alkylaryl groups, and acrylate groups, to name a few.

The water-in-silicone emulsion may be emulsified with a nonionic surfactant (emulsifier) such as, for example, polydiorganosiloxane-polyoxyalkylene block copolymers, including those described in U.S. Pat. No. 4,122,029. These emulsifiers generally comprise a polydiorganosiloxane backbone, typically polydimethylsiloxane, having side chains comprising -(EO)ₘ— and/or —(PO)ₙ— groups, where EO is ethyleneoxy and PO is 1,2-propyleneoxy, the side chains being typically capped or terminated with hydrogen or lower alkyl groups (e.g., C₁₋₆, typically C₁₋₃). Other suitable water-in-silicone emulsifiers are disclosed in U.S. Pat. No. 6,685,952. Commercially available water-in-silicone emulsifiers include those available from Dow Corning under the trade designations 3225C and 5225C FORMULATION AID; SILICONE SF-1528 available from General Electric; ABIL EM 90 and EM 97, available from Goldschmidt Chemical Corporation (Hopewell, Va.); and the SILWET series of emulsifiers sold by OSI Specialties (Danbury, Conn.).

Examples of water-in-silicone emulsifiers include, but are not limited to, dimethicone PEG 10/15 crosspolymer, dimethicone copolyol, cetyl dimethicone copolyol, PEG-15 lauryl dimethicone crosspolymer, laurylmethicone crosspolymer, cyclomethicone and dimethicone copolyol, dimethicone copolyol (and) caprylic/capric triglycerides, polyglyceryl-4 isostearate (and) cetyl dimethicone copolyol (and) hexyl laurate, and dimethicone copolyol (and) cyclopentasiloxane. In one embodiment examples of water-in-silicone emulsifiers include, without limitation, PEG/PPG-18/18 dimethicone (trade name 5225C, Dow Corning), PEG/PPG-19/19 dimethicone (trade name BY25-337, Dow Corning), Cetyl PEG/PPG-10/1 dimethicone (trade name Abil EM-90, Goldschmidt Chemical Corporation), PEG-12 dimethicone (trade name SF 1288, General Electric), lauryl PEG/PPG-18/18 methicone (trade name 5200 FORMULATION AID, Dow Corning), PEG-12 dimethicone crosspolymer (trade name 9010 and 9011 silicone elastomer blend, Dow Corning), PEG-10 dimethicone crosspolymer (trade name KSG-20, Shin-Etsu), dimethicone PEG-10/15 crosspolymer (trade name KSG-210, Shin-Etsu), and dimethicone PEG-7 isostearate.

The water-in-silicone emulsifiers typically will be present in the composition in an amount from about 0.001% to about 10% by weight, in another embodiment in an amount from about 0.01% to about 5% by weight, and in a further embodiment in an amount from 0.1% to about 2.5% by weight.

The aqueous phase of the emulsion may include one or more additional solvents, including lower alcohols, such as ethanol, isopropanol, and the like. The volatile solvent may also be a cosmetically acceptable ester such as butyl acetate or ethyl acetate; ketones such as acetone or ethyl methyl ketone; or the like.

The oil-containing phase will typically comprise from about 10% to about 99%, about 20% to about 85%, or from about 30% to about 70% by weight, based on the total weight of the emulsion, and the aqueous phase will typically comprise from about 1% to about 90%, about 5% to about 70%, or from about 20% to about 60% by weight of the total emulsion.

When applied to a biological surface, the immiscible multi-layered cosmetic of the current invention may enhance coverage and/or result in optical blurring and increased light transmittance. This reduces the appearance of dermatological signs of chronological-aging, photo-aging, hormonal-aging, and/or actinic-aging; the appearance of lines and/or wrinkles; the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; the appearance and/or depth of lines and/or wrinkles; the appearance of cellulite formation; the appearance of crow's feet; improving the appearance of rejuvenating and/or revitalizing skin, decreasing the appearance of aging skin; the appearance of skin fragility; the appearance of a loss of glycosaminoglycans and/or collagen; the appearance of estrogen imbalance; the appearance of skin atrophy; the appearance of hyperpigmentation; the appearance of skin discoloration; or any combination thereof. Also, this improves the appearance of skin tone, radiance, clarity and/or tautness, the appearance of sagging skin; the appearance of suborbital lines and/or periorbital lines; the appearance of skin firmness, plumpness, suppleness and/or softness; the appearance of procollagen and/or collagen production; the appearance of skin texture and/or retexturization; the appearance of skin barrier repair and/or function; the appearance of skin contours; the appearance of decreased skin luster and/or brightness; the appearance of dermatological signs of fatigue and/or stress; the appearance of environmental stress; the appearance of cellular aging; the appearance of skin dehydration; the appearance of elastic and/or resilient skin; the appearance of microcirculation; or any combinations thereof.

Another embodiment of the invention relates to a method of improving the aesthetic or natural appearance of a biological surface comprising applying to the biological surface, including but not limited to, keratinous tissue, skin, hair and nails, the inventive composition having the characteristics and properties described herein, in an amount effective to improve the aesthetic or natural appearance of the biological surface.

The biological surface may be any surface to which cosmetics, personal care products, dermatological, and pharmaceutical compositions are typically applied, including but not limited to skin, lips, hair, nails, and the like. The composition that is applied to skin improves or enhances the aesthetic appearance of skin by camouflaging the natural aging process, discoloration, chronic and cumulative damage to biological surfaces, and imperfections on the surface. The composition that is applied to keratinous surface or a mucous membrane improves or enhances the aesthetic appearance of the surface by enhancing the natural color and color added in the form of the pigment.

Further, in additional embodiments of the current invention, the immiscible multi-layered cosmetic composition of the current invention may improve the optical characteristics of the cosmetic. Improvements may include imparting color trueness to the cosmetic (maintaining same color across different skin types), imparting textures to the cosmetic, and the like.

In particular embodiments, the immiscible multi-layered cosmetics of the current invention provide enhanced or novel optical benefits to the skin. In each instance, the base layer is applied to and allowed to form an immiscible layer on the biological substrate prior to the application of the top layer thereon. Specific optical enhancements are discussed below.

### A. Enhanced Coverage.

In certain embodiments of the current invention, the immiscible multi-layered cosmetic composition may provide enhanced coverage of undesirable areas of the skin, i.e., hyper-pigmented areas (freckles, age spots, birthmarks, scars, etc.), blemishes (acne, hives, etc.), discolorations (dark circles under the eyes, bruises, etc.) wrinkles, etc. Further, the compositions of the current invention may provide this benefit without diminishing soft-focus provided by the top/secondary layer of the cosmetics.

As noted above, previous concealers and/or foundations often fail to provide adequate coverage for problem areas of the skin due in part to the smudging, pushed away from area of desired coverage, and mixing, dilution of coverage components, that typically occurs when make-up and/or skin care products are applied over the concealer and or foundation. It is believed, that the immiscible multi-layered cosmetic of the current invention provides for enhanced coverage because the pH dependent film forming polymer of the base layer forms an insoluble film at the pH of the skin. Thus, when the top layer, having a pH lower than or equal to the target pH of the pH dependent film-forming polymer, is applied to the skin the two do not mix.

The enhancement in coverage is illustrated by Example 2 below. In particular, Example 2A demonstrates the enhanced coverage provided by the base layer of the current invention. The immiscible multi-layer cosmetic composition of the current invention (Eudragit containing 20% particles) provided coverage of greater than about 96% in comparison to the control concealer (containing 20% particles) which provided coverage of about 90%. Further, Example 2B noted the ability of the immiscible base layer (Sample B, Eudragit containing 5.56% particles) to enhance coverage (increase in coverage from 27.8% with only foundation to 60.9%) even when the top layer does not contain colored pigments. Further, contrary to what has been experienced with prior concealers, Sample B achieved an increase in coverage with a smaller reduction in diffuse transmittance (i.e. in soft focus) than would have been expected (from 65.6 to 50.8). An increase in reflectance (increase in reflectance from 25.99 to 46.93) was also achieved when Sample B was applied as a base layer. Additional samples containing pH dependent film-forming polymers without particles were also evaluated (D+Magix and E+Magix) and the same increase in coverage was not seen.

For this particular embodiment, the base/primer layer of the cosmetic composition should further include at least one powder and/or particles. The powder and/or particles present in this embodiment of the invention can be organic pigments, inorganic pigments, organic fillers, inorganic fillers, or any combination thereof. Examples of the organic particulate pigments include azo, xanthene, quinone, lakes, especially aluminum lakes, strontium lakes, barium lakes, FD&C and D&C Red 6, Red 7, Red 30, Red 34, Yellow 5, Blue 1, or derivatives thereof, or mixtures thereof. Examples of the inorganic particulate pigments are iron oxide, especially red, yellow and black iron oxides, titanium dioxide, zinc oxide, potassium ferricyanide (K₃Fe(CN)₆), potassium ferrocyanide (K₄Fe(CN)₆), potassium ferrocyanide thhydrate (K₄Fe(CN)₆.3H₂O), and mixtures thereof. Examples of the organic fillers include nylon and starch. Examples of the inorganic fillers include talc, mica, silica, and mixtures thereof. The particles may be present in the base layer in about 0.1% to about 20%, about 1% to about 17.5%, about 1.5% to about 15%, and about 2% to 12.5%. In specific embodiments, the base layer composition may contain about 0.1%, 0.5%, 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0% and 20.0% of particles and/or powder.

Example 2A below provides an assay for assessing the coverage provided by the use of the immiscible multi-layer cosmetic compositions of the current invention. Preferably, the immiscible multi-layered compound provides coverage of at least 75%, at least 80%, at least 85%, at least 90%, and at least 95%. Further, the coverage provided by the immiscible multi-layered compositions of the current invention should be about 5%, about 10%, or about 15% better than the coverage provided in the absence of the immiscible base layer.

### B. Color Trueness

In a further embodiment of the current invention, the immiscible multi-layered cosmetic compositions of the current invention provide for enhanced color trueness. Color trueness refers to the consistent reproducibility of a color across different substrates, in the case of cosmetics across skin types. It is not uncommon for the same colored cosmetic to appear as numerous different colors when applied to a spectrum of skin types from light to dark.

The present immiscible multi-layered cosmetic permits the base/primer layer to act in a fashion similar to paint primer—opaquely covering the substrate so that the color, shade or tone of the substrate does not impact the color of the top layer. Thus, in this particular embodiment, the base/primer layer is provided with particles, powders or pigments having a refractive index (RI) of greater than or equal to about 1.40, in certain embodiments greater than about 1.5, in other embodiments greater than 1.55, and in still others greater than 1.6. In further embodiments the RI of the particles, powders or pigments may be from about 1.38 to about 3.52 or more preferably from about 1.40 to about 3.50.

Pigments having refractive indices of greater than or equal to about 1.40 include, but are not limited to, metal oxides, metal sulfides, or metal oxide mixtures, such as TiO₂, Fe₂O₃, TiFe₂O₅, Fe₃O₄, BiOCl, CoO, Co₃O₄, Cr₂O₃, VO₂, V₂O₃, Sn(Sb)O₂, SnO₂, ZrO₂, iron titanates, iron oxide hydrates, titanium suboxides (reduced titanium species having oxidation states from 2 to <4), bismuth vanadate, cobalt aluminate, and also mixtures or mixed phases of these compounds with one another or with other metal oxides. Metal sulfide coatings are preferably selected from sulfides of tin, silver, lanthanum, rare earth metals, preferably cerium, chromium, molybdenum, tungsten, iron, cobalt and/or nickel colorless or colored metal oxides, such as TiO₂, Fe₂O₃, TiFe₂O₅, Fe₃O₄, BiOCl, CoO, Co₃O₄, Cr₂O₃, VO₂, V₂O₃, Sn(Sb)O₂, SnO₂, ZrO₂, iron titanates, iron oxide hydrates, titanium suboxides (reduced titanium species having oxidation states from 2 to <4), bismuth vanadate, cobalt aluminate. TiO₂, ZrO₂, SnO₂, ZnO, BiOCl, Fe₂O₃, Fe₃O₄, Cr₂O₃, CeO₃, molybdenum oxides, CoO, Co₃O₄, VO₂, V₂O₃, NiO, V₂O₅, CuO, Cu₂O, Ag₂O, CeO₂, MnO₂, Mn₂O₃, Mn₂O₅, titanium oxynitrides, pseudobrookite, ilmenite, as well as titanium nitride, MoS₂, WS₂ or mixtures or combinations thereof. The TiO₂ here can be in the rutile or anatase modification, preferably in the rutile modification. titanium dioxide (rutile or anatase), zinc oxide and iron oxide. The refractive indices of various materials may be evaluated using a refractometer. Details with respect to the principles of refraction can be found in Optics by Eugene Hecht (Fourth Edition), 2002. Details with respect to refractive indices of materials can be found in the CRC Handbook of Chemistry and Physics, 86th Edition, 2005-2006, which is herein incorporated by reference in its entirety. In certain embodiments, titanium dioxide (RI 2.5-2.9) may be used. Powders and/or particles having refractive indices of greater than or equal to about 1.40 include, but are not limited to, nylon, PMMA, polystyrene, PTFE, barium sulfate, soda lime glass, silicon dioxide, boron nitride, kaolin, alumina, sericite, and/or talc. The powder, particles, and/or pigments with a high refractive index may be present in amounts sufficient to provide the blocking coverage desired, opaque enough to eliminate the influences of the underlying biological surface on the color of the top layer applied. In certain embodiments, the high refractive index powders, particles, and/or pigments may be present in amounts of about 0.1% to about 15%, about 1 to 12.5%, or about 2 to 10%. In specific embodiments, the base layer composition may contain about 0.1%, 0.5%, 1.0%, 2.0%, 3.0%, 4.0%, 5.0%, 6.0%, 7.0%, 8.0%, 9.0%, 10.0%, 11.0%, 12.0%, 13.0%, 14.0%, and 15.0% of high refractive index particles, pigments, and/or powder.

The top layer composition, in one embodiment, contains at least one pigment which may include organic, inorganic, lake, pearlescent, and/or effect pigment.

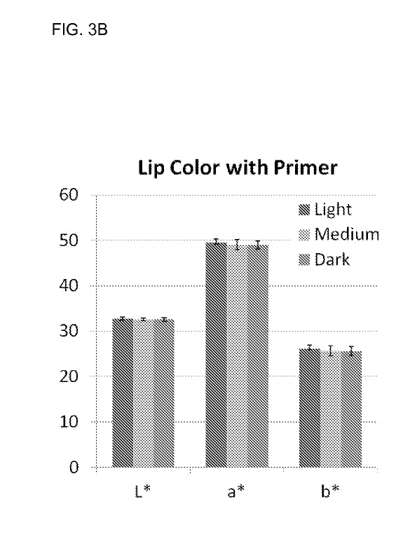

As noted in Example 3 below, the color trueness achieved by the compositions of the current invention may be evaluated by comparing the lightness and chroma of the applied cosmetics across the varying biological substrates. As shown in Example 3, color may be evaluated in the L* a* b* color space (also known as CIELAB), L* indicates lightness and a* and b* are the color directions. These aspects of color may be evaluated against the compositions applied to varying biological substrates and the L*, a*, and b* compared. If there is less than 10% variation amongst the L*, a* and b* values from the samples color trueness has been achieved, in certain embodiments it is less than 5%, in others its less than 1%, and in yet others there is no difference. As shown in FIG. 3B, when an immiscible primer is applied prior to application of the lip color the color attributes, L*, a*, and b* are substantially the same across the skin types. Thus, the color remained true.

In certain embodiments the use of the immiscible base/primer layer may permit the color to remain true as between the color within the cosmetic applicator and as on the skin, i.e. the color you see in the cosmetic container is the same color when applied to the biological substrate.

### C. Enhanced Naturalness and Coverage

In a further embodiment of the current invention, the immiscible multi-layered cosmetic of the current invention may be used to provide enhanced naturalness and coverage to the make-up. The base layer of the current invention would be provided with soft focus powders or particles that have high diffuse transmittance and low reflectance. The top layer would incorporate transparent iron oxides and fiber-shaped titanium dioxide. The transparent pigments of the top layer would allow the skin's natural tone to reflect back, and the soft focus particles of the base layer will diffuse light, blurring the imperfections. This means the makeup will not appear cakey or “masky” because the color/coverage is not opaque. And the soft focus particles in combination with the transparent pigments will allow for a soft focus and more “natural” effect.

In another embodiment, transparent pigments can be used in the first composition (base layer or primer) with or without other particles (eg soft focus particles) to provide dimension similar to how artists create paintings. Transparent pigments are light and airy barely showing on top of other colors. For example, using a transparent blue in a sky gives a greater feeling of airiniee than an opaque blue will. Artists also use transparent pigments to create a pure glazing effect, allowing the colors underneath to shine through. They have the property of transmitting light without appreciable scattering so that bodies lying beyond are seen.

The ability of a material to exhibit a soft focus effect is determined by many factors, including the chemical makeup of the material, particle size, morphology, and particle orientation. Soft focus particles of use within the current invention may have a reflective index greater than 1.4, may be spherical or non-spherical in nature and may have an average (number) particle size of about 100 nm to about 100 μm, about 500 nm to about 75 μm, about 1 μm to about 50 μm. Suitable soft focus particles for use include, but are not limited to, barium sulfate, soda lime glass, silicon dioxide, boron nitride, kaolin, alumina, sericite, talc, nylon, polyurethane, silica beads, Tospearl (Momentive), silicone crosspolymers such as Velvesil (Momentive), poly (methyl methacrylate) (PMMA), polyethylene (PE), polystyrene (PS), polytetrafluoroethylene (PTFE, e.g., Teflon®), polymethylsiloxane, cellulose beads, boron nitride, mica, polyurethane powder, sericite, silica, firmed silica, fumed alumina, talc, titanium dioxide, zinc oxide, iron oxide, silicone powder and combinations thereof. Preferable soft focus particles for use in a diffused topcoat include nylon, polyurethane, silica beads, Tospear! (Momentive), silicone elastomers such as Velvesil (Momentive), poly (methyl methacrylate) (PMMA), polyethylene (PE), polystyrene (PS), polytetrafluoroethylene (PTFE, e.g., Teflon®), polymethylsiloxane, cellulose beads, boron nitride, mica, polyure-thane powder, sericite, silica, fumed silica, fumed alumina, silicone powder and combinations thereof. In addition, effect pigments such as those disclosed in U.S. Patent Publications 20090175915 and US20120276031 may be used to achieve soft focus in the base layer. The soft focus particle is preferably present from about 0.01 weight % to about 15 weight %, about 0.5 weight % to about 12.5 weight %, and about 1 weight % to 10 weight % of the total weight of the composition.

A diffused base layer may be characterized by a percent diffused transmittance value of 20 or greater, preferably 30 or greater, and more preferably 40 or greater for an approximately 25-75 micron thin film on a glass plate. In one embodiment, the diffused base layer has a percent diffused transmittance value of about 55 or greater for an approximately 25-75 micron thin film. In another embodiment, the diffused base layer has a percent diffused transmittance value of about 60 or greater for an approximately 25-75 micron thin film. In another embodiment, the diffused base layer has a percent diffused transmittance value of about 65 or greater for an approximately 25-75 micron thin film. In one embodiment, the diffused base layer has a percent diffused transmittance value of about 64 for an approximately 25-75 micron thin film. A diffused base layer may be characterized by a percent, reflectance of 60 or less, preferably 50 or less, and more preferably 40 or less for an approximately 10 micron thin film on a glass plate. In one embodiment, the diffused base layer has a percent reflectance of about 20 or less for an approximately 25-75 micron thin film. In another embodiment, the diffused base layer has a percent reflectance of about 15 or less for an approximately 25-75 micron thin film. In another embodiment, the diffused base layer has a percent reflectance of about 10 or less for an approximately 25-75 micron thin film. In one embodiment, the diffused base layer has a percent reflectance of about 12 for an approximately 25-75 micron thin film.

The top layer may include one or more acicular pigments. In one embodiment, the acicular pigments utilized are titanium dioxide acicular pigments. In another embodiment, the acicular pigments utilized are transparent pigments. Transparent pigments may include pigments having a acicular, columnar, and or needle-like shape where in the width of the primary particle is in the range of about 1 nm to 100 nm, and the length of the primary particle is in the range of about 100 nm to 300 nm, in other embodiment having a width of about 10 nm to 60 nm and a length of about 80 nm to about 200 nm. Acicular pigments that may be used in certain aspect of the invention include, but are not limited to, (i) transparent pigments including, but are not limited to, transparent red oxides, transparent yellow oxides, transparent brown oxides, and transparent black oxides; and (ii) titanium dioxide pigments, both classes of pigments such as those provided by KOBO Products, Inc. (South Plainfield, N.J.). The acicular pigments may be present from about 0.01 weight % to about 15 weight %, about 0.5 weight % to about 12.5 weight %, and about 1 weight % to 10 weight % of the total weight of the composition.

As noted below in Example 2 the coverage and soft focus of the compositions can be evaluated using the coverage, diffuse transmittance, and reflectance protocols noted therein. However, to measure naturalness of the composition a goniospectrophotmeter (Murakami Color Research Laboratory (Model GSP-1B) Goniospectrophotometer) may be used to assess the effectiveness of soft focus from various angles. A goniospectrophotometer measures color as a function of angle. Details with respect to the principles of the use of the goniospectrophotometer can be found in Becker et al. “A Novel Method to Measure and Pre-select Functional Filler Pigments” Cosmetics & Toiletries, Vol. 127, No. 5/May 2012. Thus, control and immiscible multi-layered compositions of the current invention may be assessed for naturalness. The samples may be drawn down on (1 mL for each layer) on black Leneta cards and allowed to dry for 4 hours. The L*, a*, and b* values for the samples may be assessed at various angles from about −85° to 85°, about −75° to 75°, and from about −65° to 65° in 1°, 2°, 5°, or 10° increments. Not wishing to be bound to any particular theory, inventors believe that an increase in the intensity of transmitted light at grazing angles of the goniospectrophotometer will indicate increased efficiency in the blurring effect and therefore a more natural appearance.

One of ordinary skill in the art will appreciate that the base layer and top layer may optionally be comprised of additional colorants and pigments, and that the selection of pigments and or colorants can be made to achieve particular color combinations and/or textures.

The compositions may comprise one or more colorants or pigments to impart a desired color or effect. Examples are inorganic pigments, organic pigments, lakes, and/or dyes.

Water-soluble organic dyes include drug and cosmetic colorants that are approved for use in drugs and cosmetics by the FDA, listed in 21 C.F.R. .sctn.74.101 et seq. and including the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on.

Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe₂O₃, γ-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Non-metal oxides also contemplated to be suitable are alumina and silica, ultramarine blue (i.e., sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, and the like. Organic pigments can include, but are not limited to, at least one of carbon black, carmine, phthalocyanine blue and green pigment, diarylide yellow and orange pigments, and azo-type red and yellow pigments such as toluidine red, litho red, naphthol red and brown pigments, and combinations thereof. Dyes can include, but are not limited to, at least one of the water-soluble organic dyes listed above.

Lakes generally refer to a colorant prepared from a water-soluble organic dye, (e.g., D&C or FD&C) which has been precipitated onto an insoluble reactive or adsorptive substratum or diluent. The term “D&C” as used herein means drug and cosmetic colorants that are approved for use in drugs and cosmetics by the FDA. The term “FD&C” as used herein means food, drug, and cosmetic colorants which are approved for use in foods, drugs, and cosmetics by the FDA. Certified D&C and FD&C colorants are listed in 21 C.F.R. .§74.101 et seq. and include the FD&C colors Blue 1, Blue 2, Green 3, Orange B, Citrus Red 2, Red 3, Red 4, Red 40, Yellow 5, Yellow 6, Blue 1, Blue 2, Orange B, Citrus Red 2, and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Substrates suitable for forming lakes include, without limitation, mica, bismuth oxychloride, sericite, alumina, aluminum, copper, bronze, silver, calcium, zirconium, barium, and strontium, titanated mica, fumed silica, spherical silica, polymethylmethacrylate (PMMA), micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, nylon, silica silylate, silk powder, sericite, soy flour, tin oxide, titanium hydroxide, trimagnesium phosphate, walnut shell powder, and mixtures thereof. Suitable lakes include, without limitation, those of red dyes from the monoazo, disazo, fluoran, xanthene, or indigoid families, such as Red 4, 6, 7, 17, 21, 22, 27, 28, 30, 31, 33, 34, 36, and Red 40; lakes of yellow pyrazole, monoazo, fluoran, xanthene, quinoline, dyes or salt thereof, such as Yellow 5, 6, 7, 8, 10, and 11; lakes of violet dyes including those from the anthroquinone family, such as Violet 2. as well as lakes of orange dyes, including Orange 4, 5, 10, 11, and the like. Suitable lakes of D&C and FD&C dyes are defined in 21 C.F.R. §82.51.

The coloring agents may be optionally surface treated to for example make the particles more hydrophobic or more dispersible in a vehicle. The surface of the particles may, for example, be covalently or ionically bound to an organic molecule or silicon-based molecule or may be adsorbed thereto, or the particle may be physically coated with a layer of material. The surface treatment compound may be attached to the particle through any suitable coupling agent, linker group, or functional group (e.g., silane, ester, ether, etc). The compound may comprise a hydrophobic portion which may be selected from, for example, alkyl, aryl, allyl, vinyl, alkyl-aryl, aryl-alkyl, organosilicone, di-organosilicone, dimethicones, methicones, polyurethanes, silicone-polyurethanes, and fluoro- or perfluoro-derivatives thereof. Other hydrophobic modifiers include lauroyl lysine, Isopropyl Titanium Triisostearate (ITT), ITT and Dimethicone (ITT/Dimethicone) cross-polymers, ITT and Amino Acid, ITT/Triethoxycaprylylsilane Crosspolymer, waxes (e.g., carnauba), fatty acids (e.g., stearates), HDI/Trimethylol Hexylactone Crosspolymer, PEG-8 Methyl. Ether Triethoxysilane, aloe, jojoba ester, lecithin, perfluoroalcohol phosphate, and Magnesium Myristate (MM), to name a few.

An optional pigment component includes and alkyl silane surface-treated colorant consisting essentially of or comprising an alumina substrate (e.g., platelet shaped) and a pigment, dye, or lake bonded to the alumina substrate by an alkyl silane surface treatment. Typically, the alkyl silane will be octylsilane, and may be formed by treatment with triethoxy caprylylsilane. Non-limiting examples of such colorants include, but are not limited to, Alumina/Titanium Dioxide/Triethoxycaprylylsilane 1% (COVALUMINE™ Atlas White AS), Alumina/D&C Red Aluminum Lake CTD/Triethoxycaprylylsilane 1% (COVALUMINE™ Red Rose AS), Alumina/D&C Red Aluminum Lake CTD/Triethoxycaprylylsilane 1% (COVALUMINE™ Sonoma Red AS), Alumina/Black Iron Oxide CTD/Triethoxycaprylylsilane 1% (COVALUMINE™ Sonoma Black AS), Alumina/D&C Red #6 Aluminum Lake CTD/Triethoxycaprylylsilane 1% (COVALUMINE™ Fire Red AS), Alumina/Yellow Iron Oxide CTD/Triethoxycaprylylsilane 1% (COVALUMINE™ Sonoma Yellow AS), Alumina/D&C Blue #1 Aluminum Lake CTD/Triethoxycaprylylsilane 1% (COVALUMINE™ Astral Blue AS), Alumina/Carmine CTD/Triethoxycaprylylsilane 1% (COVALUMINE™ Campari AS), Alumina/Yellow #5 CTD/Triethoxycaprylylsilane 1% (COVALUMINE™ Sunburst AS), Alumina/Triethoxycaprylylsilane 1%, and combinations thereof, each of which is available from SENSIENT™ Cosmetic Technologies LCW.

Interference or pearl pigments may also be included. These are typically comprised of micas layered with about 50 to 300 nm films of TiO₂, Fe₂O₃, or Cr₂O₃ or the like. These include white nacreous materials, such as mica covered with titanium oxide or covered with bismuth oxychloride; and colored nacreous materials, such as titanium mica with iron oxides, titanium mica with ferric blue or chromium oxide, titanium mica with an organic pigment of the aforementioned type. If these materials are used, it is preferred that these materials are used collectively in an amount of less than 1.0 wt %. Preferably, the pearlescent component has a bismuth oxychloride based pearlescent ingredient or reflectance pearls. Bismuth oxychloride, better mimics the skin's natural reflectance, matches the skin's natural pearlescence more so than compounds such as titanium oxide. However, other pearlescent ingredients may be used. A preferred pearlescent component is called CHROMA-LITE, a combination of colored pigment bonded to BI-LITE 20 (bismuth oxychloride and mica) using calcium stearate. The CHROMA-LITE component is available in various shades/colors from Englehard Corporation (Iselin, N.J.).

In a further embodiment of the current invention, the efficacy of various active ingredients may be increased by incorporating the active agent into the base layer of the current immiscible multi-layered cosmetic composition. Given the immiscible nature of the base layer, the active will be held in close proximity to the biological substrate increasing it efficacy due to the close contact. The composition may comprise additional active ingredients having anti-aging benefits, as it is contemplated that synergistic improvements may be obtained with such combinations. Exemplary anti-aging components include, without limitation, botanicals (e.g., Butea frondosa extract); thiodipropionic acid (TDPA) and esters thereof; retinoids (e.g., all-trans retinoic acid, 9-cis retinoic acid, phytanic acid and others); hydroxy acids (including alpha-hydroxyacids and beta-hydroxyacids), salicylic acid and salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); barrier function enhancing agents (e.g., ceramides, glycerides, cholesterol and its esters, alpha-hydroxy and omega-hydroxy fatty acids and esters thereof, etc.); collagenase inhibitors; and elastase inhibitors; to name a few.

Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof.

In another embodiment, the actives may also include one or more of the following: a skin penetration enhancer, an emollient, a skin plumper, an optical diffuser, a sunscreen, an exfoliating agent, and an antioxidant.

An emollient provides the functional benefits of enhancing skin smoothness and reducing the appearance of fine lines and coarse wrinkles. Examples include isopropyl myristate, petrolatum, isopropyl lanolate, silicones (e.g., methicone, dimethicone), oils, mineral oils, fatty acid esters, cetyl ethylhexanoate, C12-15 alkyl benzoate, isopropyl isostearate, diisopropyl dimer dillinoeate, or any mixtures thereof. The emollient may be present from about 0.1 wt % to about 50 wt % of the total weight of the composition.

A skin plumper serves as a collagen enhancer to the skin. An example of a suitable skin plumper is palmitoyl oligopeptide. Other skin plumpers are collagen and/or other glycosaminoglycan (GAG) enhancing agents. When present, the skin plumper may comprise from about 0.1 wt % to about 20 wt % of the total weight of the composition.

A sunscreen for protecting the skin from damaging ultraviolet rays may also be included. In one embodiment sunscreens may include those with a broad range of UVB and UVA protection, such as octocrylene, avobenzone (Parsol 1789), octyl methoxycinnamate, octyl salicylate, oxybenzone, homosylate, benzophenone, camphor derivatives, zinc oxide, and titanium dioxide. When present, the sunscreen may comprise from about 0.01 wt % to about 70 wt % of the composition.

Suitable exfoliating agents include, for example, alpha-hydroxyacids, beta-hydroxyacids, oxaacids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. In one embodiment an exfoliating agent is glycolic acid. When present, the exfoliating agent may comprise from about 0.1 wt % to about 80 wt % of the composition.

Antioxidants scavenge free radicals from skin, protecting the skin from environmental aggressors. Examples of antioxidants that may be used in the present compositions include compounds having phenolic hydroxy functions, such as ascorbic acid and its derivatives/esters; alpha-hydroxyacids; beta-carotene; catechins; curcumin; ferulic acid derivatives (e.g. ethyl ferulate, sodium ferulate); gallic acid derivatives (e.g., propyl gallate); lycopene; reductic acid; rosmarinic acid; tannic acid; tetrahydrocurcumin; tocopherol and its derivatives (e.g., tocopheryl acetate); uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present invention may comprise an antioxidant in one embodiment from about 0.001 wt % to about 10 wt %, and in one embodiment from about 0.01 wt % to about 5 wt %, of the total weight of the composition.

Another embodiment of the invention encompasses compositions comprising a cosmetically or dermatologically acceptable formulation which is suitable for contact with living animal tissue, including human tissue, with virtually no adverse physiological effect to the user. Compositions embraced by this invention can be provided in any cosmetically and/or dermatologically suitable form, in one embodiment as a lotion or cream, but also in an anhydrous or aqueous base, as well as in a sprayable liquid form. Other suitable cosmetic product forms for the compositions of this invention include, for example, an emulsion, a cream, a balm, a gloss, a lotion, a mask, a serum, a toner, an ointment, a mousse, a patch, a pomade, a solution, a spray, a wax-based stick, or a towelette. In addition, the compositions contemplated by this invention can include one or more compatible cosmetically acceptable adjuvants commonly used and known by the skilled practitioner, such as colorants, fragrances, emollients, humectants, preservatives, vitamins, chelators, thickeners, perilla oil or perilla seed oil (WO 01/66067 to a “Method of Treating a Skin Condition,”) and the like, as well as other botanicals such as aloe, chamomile, and the like, and as further described below.

In addition to the pH-dependent film forming polymers, the inventive cosmetic compositions may comprise any number of additional ingredients, such as, but not limited to: active ingredients (e.g., cosmetic, dermatological, and/or pharmaceutical), alcohols, allergy inhibitors, amino acids, anti-acne agents (e.g., salicylic acid), anti-aging agents, antiseptics, antifungal agents, antiperspirants, analgesics, anti-hair loss agents, anti-wrinkle agents, antibacterial agents, anti-microbial agents, anti-oxidants, anti-inflammatory agents, burn healing agents, colorants (e.g., lakes, pigments, and the like), de-pigmentation agents, deodorants, dyes, emollient (e.g., glycerin, butylene glycol), excipients, fatty substances, fillers, film formers (e.g., dimethicone acrylate copolymer, ethylhexyl acrylate copolymer), fragrances, free radical scavengers, glycerin, glycerin monostearate, glycerin distearate, hair growth agents, hair conditioners, hair softeners, hair moisturizers, herbal extracts, humectants (e.g., hyaluronic acid, orotic acid, lipoprotein), insect repellants, medication, moisturizers, non-active carrier oils (e.g., triglycerides, silicone oils, mineral oils), oils, peptides, polypeptides, proteins, perfumes, pigments, preservatives, plasticizers, reflectants, sebum absorbers, skin lightening agents, sunscreens, surfactants, tanning agents, thickening agents (e.g., hydroxyethylcellulose, xanthan gum, carbomer), Vaseline, vasoconstrictors, vasodilators, vitamins (e.g., Vitamin A, Vitamin E), water, waxes, and/or combinations thereof.

The composition of the present invention may also include other cosmetic ingredients such as, but not limited to, humectants, emollients, moisturizers, anti-wrinkle ingredients, concealers, matte finishing agents, pigments, colorants, proteins, anti-oxidants, bronzers, chelating agents, emulsifiers, ultraviolet (UV) absorbing agents, oil absorbing agents, anti-foam agents, anti-tack agents, thickeners, fragrances, preservatives, anti-microbials, fungistats, neutralizing agents, vitamins, plasticizers, cohesion agents, basifying and acidifying agents, fillers, solvents, and mixtures thereof.

Additional ingredients may optionally be added to the inventive compositions as detailed below.

Fillers: Fillers can also optionally be added, in an amount from about 1% to about 20%, preferably from about 1% to about 10% by weight of the final composition. Examples of fillers include, but are not limited to, silica, PMMA, nylon, alumina, barium sulfate, or any other filler typically used in such compositions.

Film formers: Polymeric film formers include cellulosics, polyolefins, polyvinyls, polacrylates, polyurethanes, silicones, silicone acrylates, polyamides, polyesters, fluoropolymers, polyethers, polyacetates, polycarbonates, polyimides, rubbers, epoxies, formaldehyde resins, and homopolymers and copolymers of any of the foregoing.

Waxes: Waxes which may be used in the invention include, but are not limited to, linear polyethylene, microcrystalline petroleum wax, carnauba wax, lignite wax, ouricouri wax, rice bran wax, castor wax, mortar wax, stearone, acrawax, bayberry wax, castor wax, Japan wax, ozokerite, beeswax, candelilla wax, petrolatum, ceresin wax, cocoa butter, illipe butter, esparto wax, shellac wax, ethylene glycol diesters or triesters of C₁₈-C₃₆ fatty acids, cetyl palmitate, paraffin wax, hard tallow, lanolin, lanolin alcohol, cetyl alcohol, glyceryl monostearate, sugarcane wax, jojoba wax, stearyl alcohol, silicone waxes, and combinations thereof.

It is understood to those skilled in the art that any other cosmetically acceptable ingredients, i.e., those included in the CFTA Cosmetic Ingredient Dictionary, 3rd Ed., may be used.

The amount of the topical composition applied each time, the area of application, the duration of application, and the frequency of application can vary widely, depending on the specific need of the user. For example, the topical composition can be applied for a period of at least one month and at a frequency ranging from about once per week to about once per day. For another example, the topical composition is applied for a period of about six months and at a frequency ranging from about three times a week to about three times per day, and preferably about once or twice per day. The topical composition may comprise the active components at a total amount ranging from about 0.001% to about 90%, preferably from about 0.01% to about 50%, and more preferably from about 0.1% to about 30%. However, it should be noted that it is well within the purview of the skilled artisan, such as a dermatologist or other health care provider, to tailor the dosages of the topical compositions of the present invention according to specific patient needs.

In one embodiment, the ratio of methacrylic acid to methyl methacrylate is about 1:2.

In one embodiment, the acid value is from about 150 to about 200 mg KOH/g.

In one embodiment, the acid value is from about 300 to about 350 mg KOH/g.

In one embodiment, the pH-dependent film forming polymer has a target pH of greater than about 6.

In one embodiment, the pH-dependent film forming polymer has a target pH of greater than about 7.

In one embodiment, the pH-dependent film forming polymer is a polymer of poly(methacrylic acid-co-ethyl acrylate) with a ratio of methacrylic acid to ethyl acrylate of about 1:1, an acid value of from about 300 to about 350 mg KOH/g, and a weight average molar mass between about 200,000 and about 350,000 g/mol.

In one embodiment, the pH-dependent film forming polymer has a target pH of greater than about 5.5.

In one embodiment, the at least one particle or pigment is present in about 1-20% of the first composition.

In one embodiment, the pH-dependent film-forming polymer is present in an amount of about 0.1 wt % to about 15% of the total weight of the composition of the first composition.

In one embodiment, the ph-dependent film forming polymer is present in an amount of about 0.2 wt % to about 8.0% of the total weight of the first composition.

In one embodiment, the ph-dependent film forming polymer is present in an amount of about 0.5 wt % to about 5.0% of the total weight of the first composition.

In one embodiment, the pH-dependent film forming polymer is not in the form of a microcapsule.

In one embodiment, the at least one particle or pigment is titanium dioxide.

In one embodiment, the at least one particle, powder or pigment having a refractive index greater than or equal to about 1.40 is present in the first composition in about 1-15%.

In one embodiment, the transparent pigment comprises transparent yellow oxide or transparent red oxide.

In one embodiment, the arrangement comprising a human integument, a first composition comprised of a pH dependent film forming polymer having a target pH, and a second composition such that the first composition is formed as a basecoat contacting the substrate and the second composition is formed as a topcoat contacting the first composition, said first composition comprising an immiscible layer containing a pH dependent film forming polymer having a target pH and at least one soft focus particle and said second composition comprising at least one transparent pigment and having a pH less than equal to the target pH so as to provide diffuse transmittance of greater than 40%.

In one embodiment, the arrangement comprising a human integument, a first composition, and a second composition such that the first composition is formed as a basecoat contacting the substrate and the second composition is formed as a topcoat contacting the first composition, said first composition comprising an immiscible layer containing a pH dependent film forming polymer having a target pH and at least one particle or powder and said second composition having a pH less than equal to the target pH so as to provide coverage of at least about 60% and diffuse transmittance of no less than 40%.

In one embodiment, the transparent pigment comprises transparent yellow oxide, transparent red oxide, or titanium dioxide.

In one embodiment, the at least one particle or pigment is an interference or pearl pigment.

In one embodiment, a method is provided wherein each of the first and second cosmetic compositions is comprised of a particle, powder, or pigment; and wherein the multilayer cosmetic composition comprising a topcoat layer in contact with the immiscible base layer creates a visual textural finish effect.

In one embodiment, each of the topcoat layer and immiscible base layer contains a different type of effect pigment.

In one embodiment, the substantially similar tristimulus color value of the integument/base layer/topcoat combination without regard to the relative pigmentation of the underlying substrate may be characterized as color trueness.

In one embodiment, the immiscible base layer has a percent diffused transmittance value of 20 or greater.

In one embodiment, at least one of the two compositions is comprised of an interference pigment.

The following examples further illustrate various specific embodiments of the present invention, without limiting the broad scope thereof.

### EXAMPLES

### Example 1

### Immiscibility

A control concealer having the formula detailed below in Table 1 was prepared.

TABLE 1
Control Concealer Formula

| Description | Percentage |
|---|---|
| Water | 40 |
| VEEGUM | 0.4 |
| DEMINERALIZED WATER | qs |
| XANTHAN GUM | 0.2 |
| BUTYLENE GLYCOL | 6.45 |
| PRESERVATIVE | 0.6 |
| CHELATING AGENT | 0.1 |
| POE (20M) SORBITAN MONOSTEARATE | 2.75 |
| TITANIUM DIOXIDE | 12.25 |
| IRON OXIDE-YELLOW | 1.65 |
| IRON OXIDE-BLACK | 0.15 |
| COSMETIC RED OXIDE:BUTYLENE | 1.1 |
| GLYCOL GRIND 1:1 | |
| TREATED TITANIUM DIOXIDE | 4.36 |
| FUMED SILICA | 1.2 |
| PROPYLENE GLYCOL DICAPRYLATE/ | 5.8 |
| DICAPRATE | |
| ETHYLENE GLYCOL MONOSTEARATE | 2.2 |
| POP (2M) MYRISTYL ETHER PROPIONATE | 2.2 |
| SORBITAN MONOSTEARATE | 1.1 |
| ETHYLHEXYL-METHOXYCINNAMATE | 2.5 |
| CYCLOMETHICONE PENTIMER | 4.32 |
| WATER | 0.5 |
| IMIDAZOLIDINYL UREA | 0.4 |

A concealer adjusted to pH 7.5 containing Eudragit (4% in water phase) having the formula detailed below in Table 2 was prepared.

TABLE 2
Eudragit Concealer Formula

| Description | Percentage |
|---|---|
| Water | 40 |
| EUDRAGIT S100 | 4 |
| VEEGUM | 0.4 |
| Sodium Hydroxide 50% | 1.25 |
| DEMINERALIZED WATER | qs |
| XANTHAN GUM | 0.2 |
| BUTYLENE GLYCOL | 6.45 |
| PRESERVATIVE | 0.6 |
| CHELATING AGENT | 0.1 |
| POE (20M) SORBITAN MONOSTEARATE | 2.75 |
| TITANIUM DIOXIDE | 12.25 |
| IRON OXIDE-YELLOW | 1.65 |
| IRON OXIDE-BLACK | 0.15 |
| COSMETIC RED OXIDE: | 1.1 |
| BUTYLENE GLYCOL GRIND 1:1 | |
| TREATED TITANIUM DIOXIDE | 4.36 |
| FUMED SILICA | 1.2 |
| PROPYLENE GLYCOL | 5.8 |
| DICAPRYLATE/DICAPRATE | |
| ETHYLENE GLYCOL MONOSTEARATE | 2.2 |
| POP (2M) MYRISTYL ETHER PROPIONATE | 2.2 |
| SORBITAN MONOSTEARATE | 1.1 |
| ETHYLHEXYL-METHOXYCINNAMATE | 2.5 |
| CYCLOMETHICONE PENTIMER | 4.32 |
| WATER | 0.5 |
| IMIDAZOLIDINYL UREA | 0.4 |

Sodium flurorescein (Aldrich) was added at 0.1% to both the control concealer and the Eudragit concealer. A layer of each concealer—control concealer and Eudragit concealer—were applied to VitroSkin by hand (0.5 mL of concealer with 30 seconds of rubbing). After waiting 2-3 minutes, a layer of foundation, Ideal Flawless Medium Shade (Avon Products Inc.), was applied and rubbed on top each of the concealers (0.5 mL with 30 seconds of rubbing).

The coated VitroSkin was then cut using a razor blade. The samples were placed under a fluorescence microscope at 10× magnification equipped with a FITC filter to view the cross-section of the samples. Images of the cross-sections of the VitroSkin coated with the control concealer and the Eudragit concealer are shown in FIG. 1A and FIG. 1B, respectively. As shown in FIG. 1A, the image of the control concealer and foundation shows the mottled appearance of the two layers showing that components of the foundation have intermixed with the control concealer and vice versa. However, FIG. 1B, the image of the Eudragit concealer and foundation, shows a bright clear and distinct layer, the Eudragit concealer, with a darker distinct layer, the foundation, on top. FIG. 1B clearly illustrates the immiscible nature of the current two part coating.

### Example 2

### A. Coverage

In addition, the coverage of the foundation (Ideal Flawless Foundation)(Control), the above-noted control concealer and the foundation (Control), and Eudragit concealer and the foundation were compared. Each sample was drawndown (3 ml thickness per layer) on both a black Leneta card and a white Leneta card and allowed to dry for four (4) hours, after which the luminous reflectance (L*) of each of the films that were drawndown was evaluated using a Konica Minolta handheld spectrophotometer. The coverage was based on the average of these data points, and was computed using the following formula:

(Coverage)=100*L*(on black)/L*(on white).

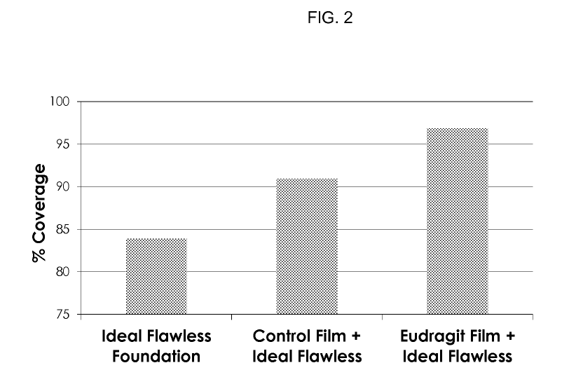

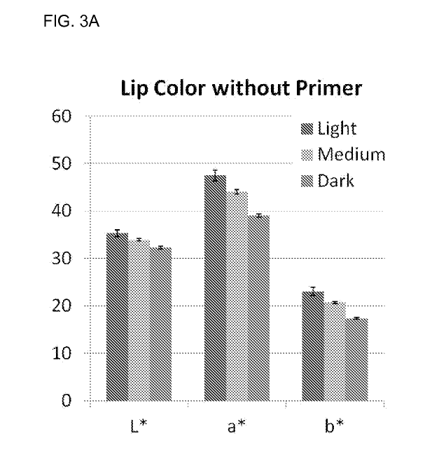

As shown within FIG. 2, The coverage (the ratio of the measured L* value of the film on a black vs. white background) increased significantly, from @ 84% with foundation alone and @ 91% with control concealer and the foundation to @ 96% with Eudragit concealer and foundation). Thus, the multi-layered immiscible cosmetic of the current invention provides enhanced coverage.

### B. Enhanced Performance of Non-Colored Cosmetics/Skin Care Products

The ability of the base/primer layer of the current invention to enhance the performance of non-colored cosmetic/skin care products (Magix, Avon Product, Inc.) was determined using the following experiment. A primer was prepared in accordance with the formulation of Table 1 above without phase F and G (pigments other than white), noted as A, B, C, and D below. Another sample comprising an alcoholic solution of Eudragit was prepared. Primers A and C contained no Eudragit, and primers B and D contained Eudragit at 4% and 1%, respectively. Sample E contained 10% Eudragit. Additionally, primers A and B had powders added as noted below in Table 3. The primer and/or primer/Magix compositions sampled are listed below in Table 3 and were evaluated for coverage, in accordance with the protocol noted above in Example 1(B) and for diffuse transmittance and reflectance, in accordance with the protocols noted below.

The diffuse transmittance and reflectance of each the samples was evaluated by drawing down the sample (3 mL for each layer) on glass plates and allowing the sample to dry at room temperature for 4 hours. Then the reflectance, total transmission and direct transmission of each of the samples was measure using an X-rite Color i7 benchtop spectrophotometer. Three (3) measurements each of the Reflection, Total Transmission and Direct Transmission were taken for each sample and were then averaged for data analysis. The diffused transmission for each sample was calculated by subtracting the direct transmission average for the sample from the total transmission average for the sample.

The results of the evaluation of coverage, diffuse transmission, and reflectance for each of the samples is noted below in Table 3. As shown in Table 3, the use of a primary layer containing powders (see Samples “A+Magix” and “B+Magix” below) boosts the coverage of Magix with a minor decrease in soft focus. The primary layer containing Eudragit (B) increased the coverage of Magix more than the control formula (A) that does not contain Eudragit, compare samples “A+Magix” and “B+Magix”. Further, the use of a primer without the addition of powders, remaining samples, did not significantly change the soft focus nor improve the coverage of the top layer.

TABLE 3

| | | | Diffuse | | Coverage |
|---|---|---|---|---|---|
| | | % | Transmittance | | (PS Test |
| Sample | Powders | Eudragit | (Soft Focus) | Reflectance | Method) |
| Magix | 18.2% TOTAL: | — | 65.58 | 25.99 | 27.8 +/− 0.8 |
| | 4% Alumina, | | | | |
| | 2.5% Fumed Silica, | | | | |
| | 2.5% Nylon, | | | | |
| | 2.5% PUR/Silica Blend, | | | | |
| | 1% PE, | | | | |
| | 0.2% BN, | | | | |
| | 5.5% Glass bubbles | | | | |
| A | 5.56% TOTAL: | — | 57.62 | 35.38 | — |
| | 4.36% TiO2 + | | | | |
| | 1.2% Fumed Silica | | | | |
| B | 5.56% TOTAL: | 4 | 56.36 | 38.18 | — |
| | 4.36% TiO2 + | | | | |
| | 1.2% Fumed Silica | | | | |
| A + | — | — | 59.08 | 39.73 | 51.0 +/− 0.5 |
| Magix | | | | | |
| B + | — | 4 | 50.82 | 46.93 | 60.9 +/− 2.0 |
| Magix | | | | | |
| C | None | — | 0 | 13.57 | — |
| D | None | 1 | 29.56 | 13 | — |
| C + | — | — | 67.66 | 25.72 | — |
| Magix | | | | | |
| D + | — | 1 | 66.84 | 28.06 | — |
| Magix | | | | | |
| E | None | 10 | 1.49 | 13.62 | — |
| E + | — | 10 | 61.97 | 28.57 | 26.6 +/− 0.7 |
| Magix | | | | | |

### Example 3

### Color Trueness

The ability of the compositions of the current invention to maintain consistency of color across skin types, i.e., color trueness, was evaluated as follows. A lip color was made in accordance with the formula noted in TABLE 4 below.

TABLE 4
Lip Color Formula

| Ingredient | Percentage |
|---|---|
| Acrylates | 38.41 |
| Copolymer | |
| Isododecane | 38.41 |
| Castor Oil | 11.59 |
| Red 7 | 2.86 |
| TiO₂ | 2.75 |
| Red 6 Ba Lake | 5.3 |
| Black Fe₂O₃ | 0.23 |
| Yellow 5 Al Lake | 0.45 |

The same lip color was then applied (3 ml drawdowns) onto three different color substrates, Leneta cards with colors to represent light, medium and dark skin tones were utilized having the following properties (TABLE 5):

TABLE 5

| | L* | a* | b* |
|---|---|---|---|
| Light | 78.76 | 6.346 | 14.526 |
| Medium | 66.722 | 11.1 | 20.24 |
| Dark | 50.496 | 15.238 | 28.046 |

A spectrophotometer was then used to measure the L*, a*, and b* of the lip color on each of the Leneta cards. FIG. 3A shows the measurements for each of the skin color Leneta cards and illustrates the variances in color between the skin tones.

Subsequently a primer containing titanium dioxide (RI 2.5-2.9) was prepared in accordance with Table 6 below.

TABLE 6
Primer Formula

| Ingredient | Percentage |
|---|---|
| Isododecane | 40 |
| Acrylates copolymer | 40 |
| Titanium dioxide | 10 |
| Castor oil | 10 |

The primer was applied to the three Leneta cards (3 ml drawdowns) with varying skin tones and allowed to dry for four (4) hours, and then the lip color (3 ml drawdown) was applied. The Leneta cards were then evaluated for L*, a*, and b* using a spectrophotometer, the results of which are shown in the bar graph of FIG. 3B. FIG. 3B clearly illustrates that the lip color appeared the same on each of the skin types when used with an immiscible base layer. The assay confirmed that using an immiscible base layer containing an opaque material (high RI) allows the subsequent pigmented lip color to be the same on every background, which cannot normally be achieved. In this case, the layers are immiscible because they were each allowed to dry completely prior to application of the lip color. Below in Table 6 is a formula for a hypothetical primer incorporating a pH dependent film-forming polymer of the current invention that is believed to provide the same benefit.

TABLE 6
Hypothetical Primer Formula

| Ingredient | Percentage |
|---|---|
| Eudragit | 1-10 |
| Water | qs |
| Tetrasodium | |
| EDTA | 0.1-1% |
| Preservative | 0.5-2% |
| Nylon | 1-10% |
| Titanium dioxide | 1-15% |

### Example 4

### Enhanced Naturalness with Coverage

An exemplary formulation for a multi-layered immiscible cosmetic according to the current invention is provided below.

TABLE 7
Makeup Layer with Transparent Pigments

| Description | % |
|---|---|
| DEMINERALIZED WATER | 68.84 |
| DISODIUM EDTA-TECH.GRADE | 0.2 |
| GLYCERIN | 7 |
| Polyacrylate Crosspolymer-6 | 1 |
| GLYCERIN | 3 |
| SODIUM HYDROXIDE SOLUTION 50% | 0.1 |
| Titania Fiber | 0-10% |
| Transparent yellow grind | 0-10% |
| Transparent red grind | 0-10% |
| Black Iron Oxide | 0-10% |
| SHEA BUTTER (BUTYROSPERMUM | 5 |
| PARKII)-LIQUID | |
| CETYL/STEARYL ALCOHOL (60/40) | 0.5 |
| PEG-40 STEARATE | 0.5 |
| ISONONYL/ISONONANOATE | 2 |
| DIMETHICONE—(2500-2900 CST) | 0.5 |
| DIAZOLIDINYL UREA-100% | 0.5 |

TABLE 8
Primer Layer with Soft Focus Particles & Eudragit

| Description | % |
|---|---|
| DEMINERALIZED WATER | 73.7 |
| DISODIUM EDTA-TECH.GRADE | 0.2 |
| GLYCERIN | 10 |
| SODIUM HYDROXIDE SOLUTION 50% | 0.1 |
| EUDRAGIT S100 | 1 |
| Polyacrylate Crosspolymer-6 | 1 |
| Soft Focus Particles/Powders | 5 |
| SHEA BUTTER (BUTYROSPERMUM | 5 |
| PARKII)-LIQUID | |
| CETYL/STEARYL ALCOHOL (60/40) | 0.5 |
| PEG-40 STEARATE | 0.5 |
| ISONONYL/ISONONANOATE | 2 |
| DIMETHICONE—(2500-2900 CST) | 0.5 |
| DIAZOLIDINYL UREA-100% | 0.5 |

The invention having been described by the foregoing description of the preferred embodiments, it will be understood that the skilled artisan may make modifications and variations of these embodiments without departing from the spirit or scope of the invention as set forth in the following claims.

1. A method comprising:
a. applying a basecoat of a first cosmetic composition to a substrate
i. wherein the first cosmetic composition has a pH greater than or equal to a target pH;
ii. wherein the first cosmetic composition is comprised of at least one particle or powder; and
iii. wherein the first cosmetic composition is comprised of a cosmetically or pharmaceutically acceptable vehicle;
b. forming an immiscible base layer on application of the first cosmetic composition to the substrate; and
c. applying a topcoat of a second cosmetic composition to the immiscible base layer to produce a multilayer cosmetic composition,
i. wherein the second cosmetic composition has a pH less than or equal to the target pH.

2. The method of claim 1, wherein the second cosmetic composition forms a topcoat layer in contact with the immiscible base layer.

3. The method of claim 1, wherein the substrate is a human integument.

4. The method of claim 3, wherein the multilayer cosmetic composition achieves
i. at least 50% coverage; and
ii. diffuse transmittance of no less than 20%.

5. The method of claim 1, wherein the first cosmetic composition is further comprised of a transparent pigment.

6. The method of claim 1, wherein the first cosmetic composition comprises at least one active agent.

7. The method of claim 1,
a. wherein the substrate is at least one black and at least one while Leneta card, and
b. wherein, when the method is practiced by sequential drawdown (3 ml thickness per layer) of the first cosmetic composition and second composition on each of a black Leneta card and a white Leneta card and allowed to dry for four (4) hours, the coverage of the multilayer cosmetic film is evaluated in accordance with Example 2 herein and is at least 90%.

8. A method comprising:
a. applying a basecoat of a first cosmetic composition to a substrate,
i. wherein the first cosmetic composition has a pH greater than or equal to a target pH;
ii. wherein the first cosmetic composition is comprised of at least one particle, powder, or pigment having a refractive index of at least 1.4; and
iii. wherein the first cosmetic composition is comprised of a cosmetically or pharmaceutically acceptable vehicle;
b. forming an immiscible base layer on application of the first cosmetic composition to the substrate; and
c. applying a topcoat of a second cosmetic composition to the immiscible base layer to produce a multilayer cosmetic composition,
i. wherein the second cosmetic composition has a pH less than or equal to the target pH; and
ii. wherein the second cosmetic composition is comprised of a colorant;
d. wherein the applied multilayer cosmetic composition, when formed on substrates of various color, will yield a substantially similar tristimulus color value of the integument/base layer/topcoat combination without regard to the relative pigmentation of the underlying substrate.

9. The method of claim 6, wherein the substrate is a human integument.

10. The method of claim 3, wherein the human integument is skin or lips.

11. A method comprising:
a. applying a basecoat of a first cosmetic composition to a substrate
i. wherein the first cosmetic composition has a pH greater than or equal to a target pH;
ii. wherein the first cosmetic composition is comprised of at least one particle or powder; and
iii. wherein the first cosmetic composition is comprised of a cosmetically or pharmaceutically acceptable vehicle;
b. forming an immiscible base layer on application of the first cosmetic composition to the substrate; and
c. applying a topcoat of a second cosmetic composition to the immiscible base layer to produce a multilayer cosmetic composition,
i. wherein the second cosmetic composition has a pH less than or equal to the target pH;
ii. wherein the second cosmetic composition is comprised of an acicular pigment; and
iii. wherein the multilayer cosmetic composition has a diffuse transmittance of no less than 20%.

12. The method of claim 9, wherein the acicular pigment is a transparent pigment.

13. The method of claim 9, wherein the acicular pigment is a titanium dioxide acicular pigment.

14. (canceled)

15. (canceled)

16. (canceled)

17. (canceled)

18. (canceled)

19. The method of claim 1, wherein said a pH-dependent film-forming polymer is a polymer of poly(methacrylic acid-co-methyl methacrylate) with a ratio of methacrylic acid to methyl methacrylate of about 1:1 to about 1:2, an acid value of from about 150 to about 350 mg KOH/g, and a weight average molar mass between about 100,000 and about 150,000 g/mol.

20. The method of claim 19, wherein said ratio of methacrylic acid to methyl methacrylate is about 1:1.
